(19) United States
(12) Patent Application Publication
(10) Pub. No.: US 2020/0141864 A1
(43) Pub. Date: May 7, 2020

(54) IDENTIFICATION OF MICROORGANISMS USING REFLECTION INFRARED SPECTROSCOPY

(71) Applicant: THE ROYAL INSTITUTION FOR THE ADVANCEMENT OF LEARNING/MCGILL UNIVERSITY, Montreal (CA)

(72) Inventors: Ashraf A. ISMAIL, Westmount (CA); Jacqueline SEDMAN, Kirkland (CA); Pierre LEBEL, Saint-Laurent (CA)

(21) Appl. No.: 16/625,004

(22) PCT Filed: Jun. 27, 2018
(86) PCT No.: PCT/CA2018/050794

§ 371 Date: Dec. 20, 2019

Related U.S. Application Data
(60) Provisional application No. 62/527,327, filed on Jun. 30, 2017.

(51) Int. Cl. G01N 21/35; C12Q 1/04

(52) U.S. Cl. CPC G01N 21/35; G01N 2021/3595; C12Q 1/04

(57) ABSTRACT

The present disclosure presents methods and systems for the spectral identification of microorganisms using reflection infrared spectroscopy. A background spectrum is acquired to measure a water vapor level of an ambient atmosphere in the absence of a sample. The sample containing the microorganism is brought into contact with an infrared reflective substrate, the sample has intact microbial cells. Spectral data is acquired from the sample using reflection infrared spectroscopy no more than a predetermined time after having acquired the background spectrum. The background spectrum and the spectral data are combined thereby producing modified spectral data. The microorganism is characterized using the modified spectral data.

### TECHNICAL FIELD

The present disclosure relates generally to analyzing microorganisms using spectral data obtained from infrared spectroscopy, and particularly to microbial differentiation and identification using reflection IR spectroscopy.

### BACKGROUND OF THE ART

The use of infrared spectroscopy for microbial differentiation and identification dates back to 1954. The feasibility of such application of infrared spectroscopy was substantially enhanced by the advent of Fourier transform infrared (FTIR) spectroscopy and has been extensively investigated by numerous research groups over the past three decades. Taken together, this body of research indicates that the infrared spectra of pure microbial colonies serve as whole-organism fingerprints that are specific down to the subspecies level of taxonomic classification. However, the reliability of infrared spectroscopy as a means of microbial identification is dependent upon all the conditions employed in the identification procedure, beginning with growth of the microorganisms on culture media to obtain pure colonies and followed by sample preparation for infrared spectroscopic measurement, which entails the deposition of microbial cells, taken from one or more pure colonies, as a thin film on a suitable substrate.

FTIR spectra of microorganisms are commonly acquired in the transmission mode, although various other techniques such as attenuated total reflectance (ATR) and diffuse reflectance spectroscopy (DRIFT) have also been employed. For spectra acquired in the transmission mode, spectral reproducibility depends mainly on the uniformity of the sample (sample homogeneity, particle size) and sample thickness (or path length). Sample non-uniformity leads to baseline variations owing to the scattering, diffraction, and refraction that occur as the IR beam passes through the sample, whereas variations in sample thickness result in variations in band intensity, although consistency in relative peak intensities is maintained.

There is therefore a need for improved methods for identifying microorganisms using spectral data.

### SUMMARY

The present disclosure presents methods and systems for the spectral identification of microorganisms using reflection infrared spectroscopy.

In accordance with a first broad aspect, there is provided a method for spectral identification of a microorganism. The method comprises acquiring a background spectrum to measure a water vapor level of an ambient atmosphere in the absence of a sample, bringing the sample containing the microorganism into contact with an infrared reflective substrate, the sample having intact microbial cells, acquiring spectral data from the sample using reflection infrared spectroscopy no more than a predetermined time after having acquired the background spectrum, combining the background spectrum and the spectral data, thereby producing modified spectral data, and characterizing the microorganism using the modified spectral data.

In some embodiments, the infrared reflective substrate is a substrate coated with a material having an infrared-reflecting property.

In some embodiments, the infrared reflective substrate is any one of an indium-tin-oxide coated substrate, a metal-coated substrate, and a metal oxide-coated substrate.

In some embodiments, the infrared reflective substrate is comprised of a material having an infrared-reflecting property.

In some embodiments, the infrared reflective substrate is any one of a steel substrate, an E-glass substrate, a metal substrate, and a metal oxide substrate.

In some embodiments, the infrared reflective substrate is any one of an indium-tin-oxide coated substrate, a steel substrate, an E-glass substrate, a metal substrate, a metal-coated substrate, a metal oxide substrate, and a metal oxide-coated substrate.

In some embodiments, the infrared reflective substrate is a matrix-assisted laser desorption/ionization time of flight mass spectrometry (MALDI-TOF MS) slide.

In some embodiments, the background spectrum is acquired in a path between an infrared source and an infrared detector defined for acquisition of the spectral data while the infrared reflective substrate is without the sample.

In some embodiments, the spectral data is acquired from the sample prior to or after having added a MALDI-TOF MS chemical matrix thereto.

In some embodiments, combining the background spectrum and the spectral data comprises computing a logarithm of the spectral data divided by the background spectrum to obtain the modified spectral data.

In some embodiments, the method further comprises comparing a water vapor level of the modified spectral data to a first threshold and rejecting the modified spectral data when the water vapor level is above the first threshold.

In some embodiments, the method further comprises comparing a water content level of the modified spectral data to a second threshold and rejecting the modified spectral data when the water content level is below the second threshold.

In some embodiments, the method further comprises comparing a biomass of the sample, extracted from the modified spectral data, to a third threshold and rejecting the modified spectral data when the biomass is below the third threshold.

In some embodiments, the sample has a limited free water content and an intact associated and bound water content.

In some embodiments, the sample has a water activity of less than 0.999.

In some embodiments, the method further comprises applying a vacuum to the sample on the infrared reflective substrate prior to acquiring the spectral data from the sample using reflection infrared spectroscopy.

In some embodiments, acquiring spectral data from the sample comprises acquiring spectral data from the sample in the absence of a drying treatment being applied to the sample.

In some embodiments, acquiring spectral data from the sample comprises acquiring spectral data from the sample in the absence of a reagent being applied to the sample for reducing water content of the sample.

In some embodiments, acquiring the spectral data from the sample comprises acquiring Fourier transform infrared spectrum.

In some embodiments, the method further comprises using the modified spectral data to enhance the characterization of the microorganism by matrix-assisted laser desorption/ionization time of flight mass spectrometry.

In some embodiments, characterizing the microorganism comprises comparing the modified spectral data to reference data to determine an identity of the microorganism.

In accordance with a second broad aspect, there is provided a system for spectral identification of a microorganism. The system comprises a processing unit and a non-transitory computer-readable memory having stored thereon program instructions. The program instructions are executable for receiving a background spectrum to measure a water vapor level of an ambient atmosphere in the absence of a sample; receiving spectral data from the sample in contact with an infrared reflective substrate using reflection infrared spectroscopy and acquired no more than a predetermined time after having acquired the background spectrum, the sample containing the microorganism and having intact microbial cells; combining the background spectrum and the spectral data, thereby producing modified spectral data; and characterizing the microorganism using the modified spectral data.

In some embodiments, the infrared reflective substrate is a substrate coated with a material having an infrared-reflecting property.

In some embodiments, the infrared reflective substrate is any one of an indium-tin-oxide coated substrate, a metal-coated substrate, and a metal oxide-coated substrate.

In some embodiments, the infrared reflective substrate is comprised of a material having an infrared-reflecting property.

In some embodiments, the infrared reflective substrate is any one of a steel substrate, an E-glass substrate, a metal substrate, and a metal oxide substrate.

In some embodiments, the infrared reflective substrate is any one of an indium-tin-oxide coated substrate, a steel substrate, an E-glass substrate, a metal substrate, a metal-coated substrate, a metal oxide substrate, and a metal oxide-coated substrate.

In some embodiments, the infrared reflective substrate is a MALDI-TOF MS slide.

In some embodiments, the background spectrum is acquired in a path between an infrared source and an infrared detector defined for acquisition of the spectral data while the infrared reflective substrate is without the sample.

In some embodiments, the spectral data is acquired from the sample prior to or after having added a MALDI-TOF MS chemical matrix thereto.

In some embodiments, combining the background spectrum and the spectral data comprises computing a logarithm of the spectral data divided by the background spectrum to obtain the modified spectral data.

In some embodiments, the program instructions are further executable for comparing a water vapor level of the modified spectral data to a first threshold and rejecting the modified spectral data when the water vapor level is above the first threshold.

In some embodiments, the program instructions are further executable for comparing a water content level of the modified spectral data to a second threshold and rejecting the modified spectral data when the water content level is below the second threshold.

In some embodiments, the program instructions are further executable for a biomass of the sample, extracted from the modified spectral data, to a third threshold and rejecting the modified spectral data when the biomass is below the third threshold.

In some embodiments, the sample has a limited free water content and an intact associated and bound water content.

In some embodiments, the sample has a water activity of less than 0.999.

In some embodiments, the program instructions are further executable for applying a vacuum to the sample on the infrared reflective substrate prior to acquiring the spectral data from the sample using reflection infrared spectroscopy.

In some embodiments, acquiring spectral data from the sample comprises acquiring spectral data from the sample in the absence of a drying treatment being applied to the sample.

In some embodiments, acquiring spectral data from the sample comprises acquiring spectral data from the sample in the absence of a reagent being applied to the sample for reducing water content of the sample.

In some embodiments, acquiring the spectral data from the sample comprises acquiring Fourier transform infrared spectrum.

In some embodiments, the program instructions are further executable for using the modified spectral data to enhance the characterization of the microorganism by matrix-assisted laser desorption/ionization time of flight mass spectrometry.

In some embodiments, characterizing the microorganism comprises comparing the modified spectral data to reference data to determine an identity of the microorganism.

In accordance with another broad aspect, there is provided a system for spectral identification of a microorganism. The system comprises a processing unit and a non-transitory computer-readable memory having stored thereon program instructions. The program instructions are executable for acquiring a background spectrum to measure a water vapor level of an ambient atmosphere in the absence of a sample, acquiring spectral data from the sample using reflection infrared spectroscopy no more than a predetermined time after having acquired the background spectrum, the sample having been brought into contact with the infrared reflective substrate and having intact microbial cells, combining the background spectrum and the spectral data, thereby producing modified spectral data, and characterizing the microorganism using the modified spectral data.

### BRIEF DESCRIPTION OF THE DRAWINGS

Further features and advantages of the present invention will become apparent from the following detailed description, taken in combination with the appended drawings, in which:

FIG. 1 is a diagram of an example setup for reflection infrared spectroscopy of a microorganism;

FIG. 2 is a flowchart of an example embodiment for a method of identifying microorganisms using reflection IR spectroscopy;

FIG. 3 is an example of a background spectrum;

FIG. 4 is an example of a modified background spectrum;

FIG. 5 is an enlarged portion of the background spectrum of FIG. 4;

FIG. 6 is an example of a multi-tier classification strategy;

FIG. 7 is another example of classification;

FIG. 8 is an example of modified spectral data to validate water vapor level, water content of the sample, and biomass of the sample;

FIG. 9 illustrates modified spectral data that is non-compliant with regards to water vapor level and water content;

FIG. 10 illustrates modified spectral data that is non-compliant with regards to biomass of the sample;

FIG. 11 is an example database structure for Gram-positive bacteria;

FIG. 12 is an example of discrimination between enterococci and staphylococci;

FIG. 13 is an example of discrimination between Enterococcus faecalis and E. faecium;

FIGS. 14A-14B are examples of discrimination between vancomycin-resistant enterococci (VRE) and vancomycin-sensitive enterococci (VSE);

FIG. 15 is an example of discrimination between methicillin-resistant Staphylococcus aureus (MRSA) and methicillin-sensitive Staphylococcus aureus (MSSA);

FIG. 16 is a dendrogram showing differentiation between MRSA and MSSA by hierarchical cluster analysis of spectral data acquired by reflection infrared spectroscopy;

FIG. 17 is a dendrogram showing differentiation between Enterococcus faecalis and E. faecium by hierarchical cluster analysis of spectral data acquired by reflection infrared spectroscopy;

FIG. 18 is an example system for spectral identification of microorganisms using reflection IR spectroscopy;

FIG. 19 is an example embodiment for a microorganism identification device;

FIG. 20 is an example embodiment of an application running on the microorganism identification device of FIG. 19;

FIG. 21 is an example of results obtained using a feature selection algorithm (FSA);

FIGS. 22A-22B illustrate the differentiation between Escherichia coli and Shigella species and the identification of bacteria isolated from positive blood culture tubes as E. coli;

FIG. 23 is an example of identification of a yeast clinical isolate;

FIG. 24 is an example of the differentiation of antibiotic-sensitive and antibiotic-resistant microorganisms;

FIG. 25 is an example of differentiation between Gram-positive and Gram-negative microorganisms, including microorganisms extracted directly from positive blood cultures;

FIG. 26 is an example of the correct classification of clinical isolates of vancomycin-resistant E. faecium;

FIGS. 27A-27B are examples of reflection IR spectroscopy performed on MALDI-TOF MS slides;

FIG. 28 is another example of reflection IR spectroscopy performed on MALDI-TOF MS slides for differentiating E. coli O157 from other serotypes of Shiga toxin-producing E. coli;

FIG. 29 is an example of reflection focal-plane-array (FPA) FTIR spectroscopy performed on MALDI-TOF MS slides;

FIG. 30 illustrates the correlation of spectral features in the reflection-FTIR spectra to m/z lines derived from MALDI-TOF MS; and

FIG. 31 illustrates an example of reflection-FTIR spectra coupled with MALDI-TOF MS for enhancing the discrimination between E. coli and Shigella.

It will be noted that throughout the appended drawings, like features are identified by like reference numerals.

### DETAILED DESCRIPTION

There are described herein methods and systems for spectral identification of a microorganism. The microorganism may be any microscopic living organism that is single-celled, such as but not limited to bacteria, archaea, yeasts, fungi, and molds. A sample of the microorganism is provided on an infrared reflective substrate. The sample contains intact microbial cells having a limited water content level. No drying treatments are applied to the sample, and no reagents are used to reduce or eliminate the original water content of the sample during the sample preparation time. Free water mostly evaporates as soon as the sample is placed on the infrared reflective substrate, while associated water and bound water remain.

In some embodiments, a vacuum may be applied post-deposition of the microorganism on the infrared reflective substrate for the purpose of removing any remaining free water and associated water in a consistent manner. The infrared spectrum may thus be recorded while the microorganism is under vacuum.

Spectral identification is thus performed based on characteristic spectral fingerprints of intact, whole organisms, with minimal post-culture sample preparation required. Spectral databases of well-characterized strains and multivariate statistical analysis techniques are used to identify unknowns by matching their spectra against those in a reference spectral database.

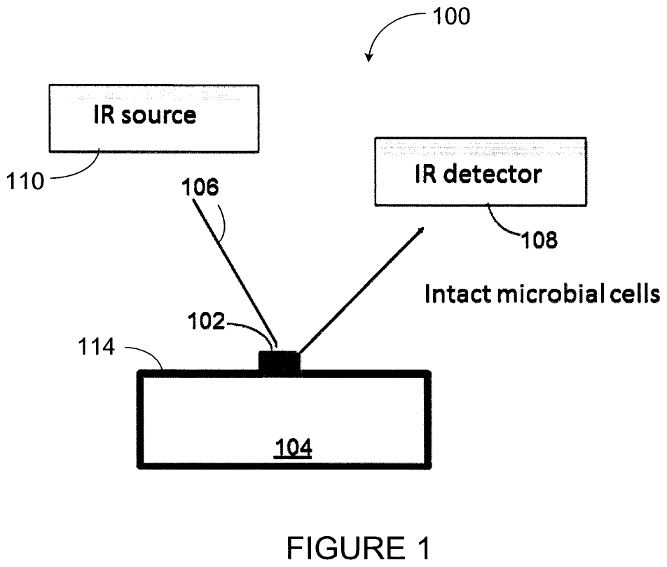

FIG. 1 illustrates an example setup 100 used for spectral identification of a microorganism. The sample 102 sits on a surface 114 of an infrared reflective substrate 104. The sample 102 may be taken from any known culture medium without breaking the culture medium surface and deposited onto the infrared reflective substrate 104 using a transfer device (not shown) such as a sterile toothpick or loop.

The sample 102 may be obtained from a microbial culture, a blood culture, bodily fluids (such as urine and pus, nasal and wound swabs), food, water, air, and the like. The size of the sample 102 should be sufficient to cover an area of the infrared reflective substrate 104. In some embodiments, the sample 102 is sized to be about one tenth ( 1/10) to three millimeters in diameter. Other sample sizes may also be used.

The surface of infrared reflective substrate 104 is made of a material having an infrared-reflecting property, so that reflection of a beam 106, at an angle, off surface 114 in contact with the sample 102 returns the reflected IR beam toward an infrared detector 108 subsequent to passing through the sample 102. The beam 106 is emitted by an IR source 110. In some embodiments, the infrared reflective substrate 104 is a substrate material (plastic or glass) coated with an indium-tin-oxide coating. Other materials, such as steel, metallic surfaces, or E-glass may also be used for the infrared reflective substrate 104. A beam 106 of infrared light is passed through the sample 102 and reflects from the infrared reflective substrate 104 passing back through the sample 102 in such a way that it reflects at least once off the reflective surface 114 in contact with the sample 102. Various optical components, such as lenses and/or mirrors, may be used to direct the beam 106 from a light source 110 to the infrared reflective substrate 104 and back towards the detector 108.

In some embodiments, the infrared reflective substrate 104 is mounted inside an infrared spectrometer, which may be a Fourier transform infrared (FTIR) spectrometer or a dispersive spectrometer. Any device that can acquire an infrared spectrum in the spectral region between 4000 and 400 wavenumbers and that can be coupled with a reflection accessory, such as devices that are filter-based, variable filter array-based, FTIR-based, and quantum cascade laser (QCL)-based, may be used. The light source 110 may be an infrared light source configured to emit light at one or more wavelengths, and the detector 108 may be an infrared detector configured for detecting the reflected beam 112 at a single detection point or a plurality of detection points corresponding to different regions of the sample 102. In some embodiments, the infrared spectrometer is an FTIR spectrometer operating in rapid-scan mode and having an infrared microscope and a focal-plane-array (FPA) detector, such as a 32×32 array of detector elements, referred to herein as an FPA-FTIR spectrometer. In some embodiments, the infrared spectrometer is a dispersive spectrometer that employs a linear variable filter and a pyroelectric detector array.

In some embodiments, the infrared reflective substrate 104 is a MALDI-TOF MS (matrix-assisted laser desorption/ionization time-of-flight mass spectrometry) slide. Indeed, and as will be described in more detail below, the same slide or substrate may be used for both MALDI-TOF MS and reflection IR spectroscopy. In some embodiments, the reflection IR spectral data is acquired from the MALDI-TOF MS slide after the addition of the MALDI-TOF MS chemical matrix. The reflection IR spectral data may also be acquired before the addition of the MALDI-TOF MS chemical matrix. Accordingly, the standard operating procedure (SOP) for MALDI-TOF MS analysis is not affected. In some embodiments, the system used for performing the analysis is an FPA-FTIR or single-detector raster imaging device combined with MALDI-TOF MS hardware as a hybrid system. Alternatively, an independent imaging device for reflection IR measurements may be used.

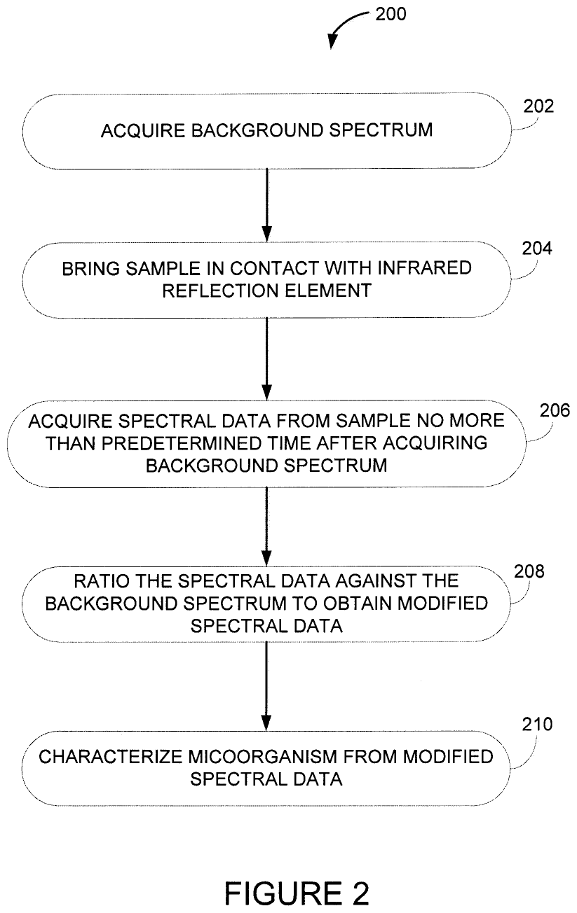

Referring to FIG. 2, there is illustrated a method 200 for identification of a microorganism using the setup 100 of FIG. 1. At step 202, a background spectrum is acquired. The background spectrum measures a water vapor level of the ambient atmosphere in the path between the light source 110 and the detector 108. For example, the beam 106 may be measured by the detector 108 when the surface 114 of the infrared reflective substrate 104 is without the sample.

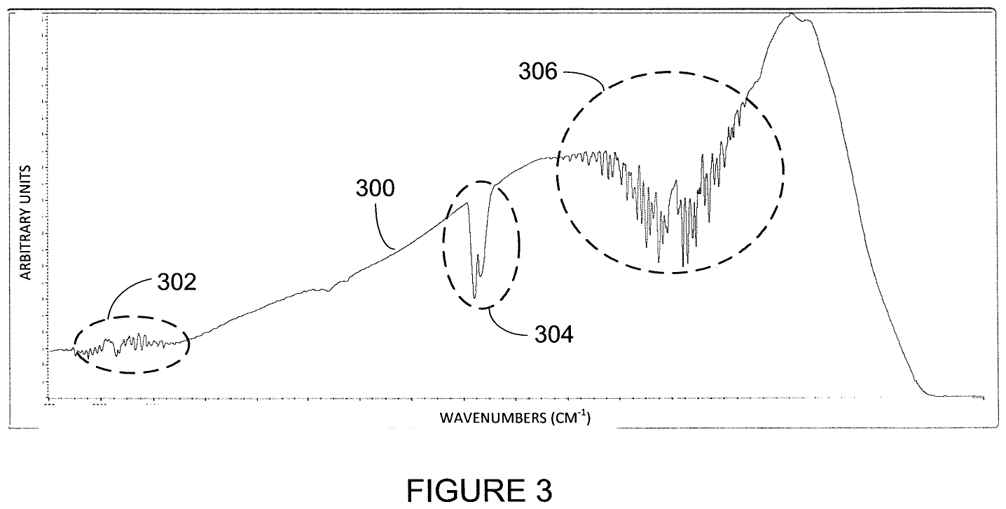

FIG. 3 is an example of a background spectrum 300. The spectrum 300 was acquired off a plastic substrate coated with an indium-tin-oxide coating. The region 302 of the background spectrum 300 is representative of the water vapor in the atmosphere. The region 304 is representative of the CO₂ in the atmosphere. The region 306 is representative of the water vapor in the atmosphere. The signal 300 was acquired by co-adding 64 scans taken during 45 seconds. Note that fewer scans, such as 4, 16, and 32, may be used, and more scans, such as 128 and 256 may be used.

Referring back to FIG. 2, once the background spectrum has been acquired, as per step 202, the sample 102 is brought into contact with the infrared reflective substrate 104 using any automated and/or manual means in a manner without compromising the integrity of the intact microbial cells, as per step 204. As explained above, the sample 102 may be transferred onto the infrared-reflective substrate 104 using any type of transfer device.

At step 206, spectral data from the sample is acquired no more than a predetermined amount of time after bringing the sample 102 into contact with the infrared reflective substrate 104 without compromising the integrity of the intact microbial cells. In some embodiments, the predetermined amount of time is less than or equal to one minute. In some embodiments, the predetermined amount of time is selected from a range of about two minutes to about five seconds. In some embodiments, the predetermined amount of time is the minimal time it takes to swab the culture medium, apply the sample to the reflective substrate 104, and press scan on the spectrometer. When automated, the sample 102 may be kept at a very close distance to the reflective substrate 104 without being in contact therewith while the background spectrum is acquired, followed by immediate contact of the sample 102 with the reflective substrate 104 and acquisition of the spectral data. A full spectral range from 4000 cm⁻¹ to 400 cm⁻¹ may be acquired, even though spectral data from one or more narrower spectral regions may be employed for the purpose of enhancing reproducibility and accuracy of bacterial differentiation. In some embodiments, if it is desired to access spectral regions partially masked by H₂O absorption, for example, the spectral region between 1700 and 1600 cm⁻¹, the H₂O in the sample may be replaced by deuterium oxide (D₂O).

At step 208, the background spectrum and the spectral data are combined to obtain the modified spectral data. Combining the background spectrum and the spectral data may also be viewed as performing a ratio of the spectral data against the background spectrum. The acquisitions are combined to obtain a transmittance spectrum that is then used to produce an absorbance spectrum “A”. The time between the two acquisitions, namely of the background spectrum and the spectral data from the sample, is limited in order to prevent evaporation of the water content from the sample, and to ensure as close a match as possible of the water vapor content of the ambient atmosphere between the two acquisitions. As such, when the background spectrum and the spectral data are combined, water vapor bands are effectively eliminated from the spectral data.

In some embodiments, combining the background spectrum and the spectral data comprises dividing the sample data by the background data (to obtain the transmittance spectrum) and taking a logarithm of the result (to obtain the absorbance spectrum):

A=−log₁₀(sample/background)

The result (“A”) may be viewed as modified spectral data, as the water vapor bands from the sample spectral data have been removed, and it forms the basis of the analysis performed in order to characterize the microorganism, as per step 210.

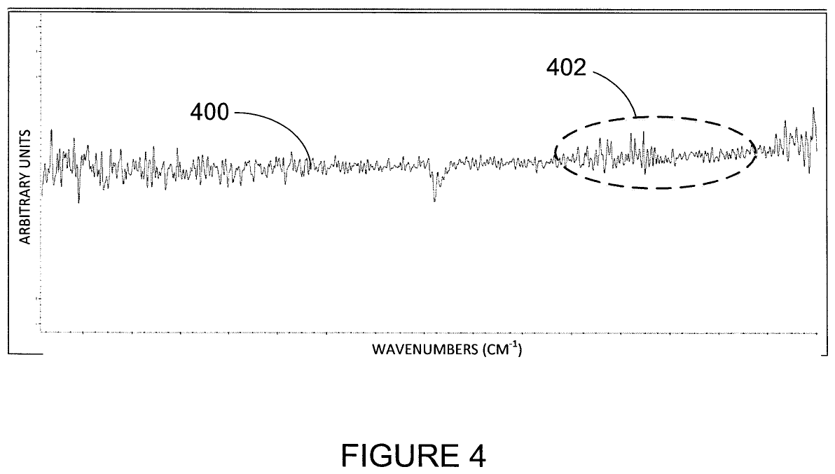

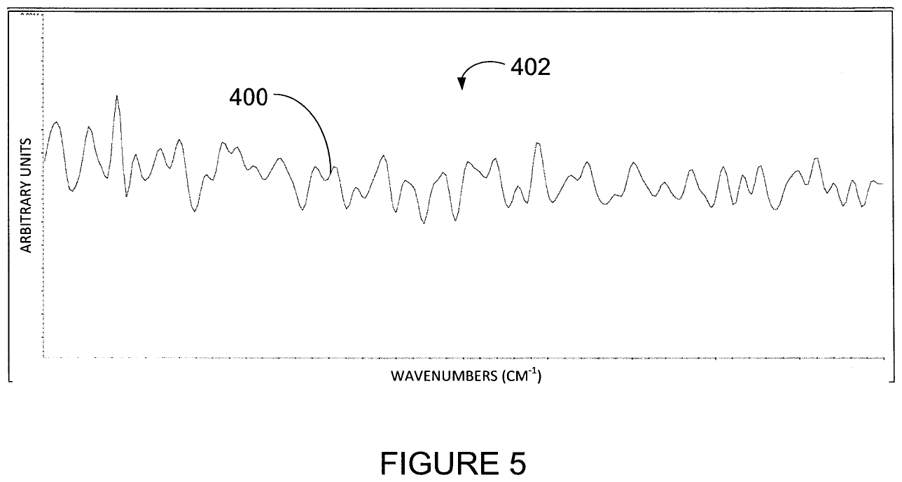

FIG. 4 is an example of modified spectral data 400 acquired in the absence of a sample. The region 402 shows a peak-to-peak noise level of less than 0.0005 absorbance units. Note the absence of water vapor bands across the entire spectrum. FIG. 5 is an enlarged view of the spectrum 400 inside region 402. The peak-to-peak noise level is 0.002 absorbance units for the range of 1406.765 cm⁻¹ to 957.953 cm⁻¹. The root-mean-square (RMS) noise level is 3.952*10⁻⁴.

In some embodiments, step 210 of the method 200 is performed as described in U.S. Pat. No. 9,551,654, the contents of which are incorporated by reference. For example, at least one multi-pixel spectral image of the sample is obtained, wherein each pixel of the image has a corresponding spectrum, and one or more spectra is selected from the spectral image based on one or more spectral characteristics of the corresponding spectrum. The microorganism may be identified by comparing the one or more selected spectra with spectra of reference microorganisms from a database. The modified spectral data is compared to those in the spectral databases containing spectra of pre-characterized isolates. Single or multiple multivariate methods may be employed for the identification of the isolate. Among the multivariate methods are hierarchical cluster analysis (HCA), principal component analysis (PCA), partial least squares (PLS), and spectral search which generate a similarity match between the spectra of unknown isolate and a near identical spectrum in the spectral database. It should be noted that selected spectral regions rather than the full spectrum may be employed in the identification procedure.

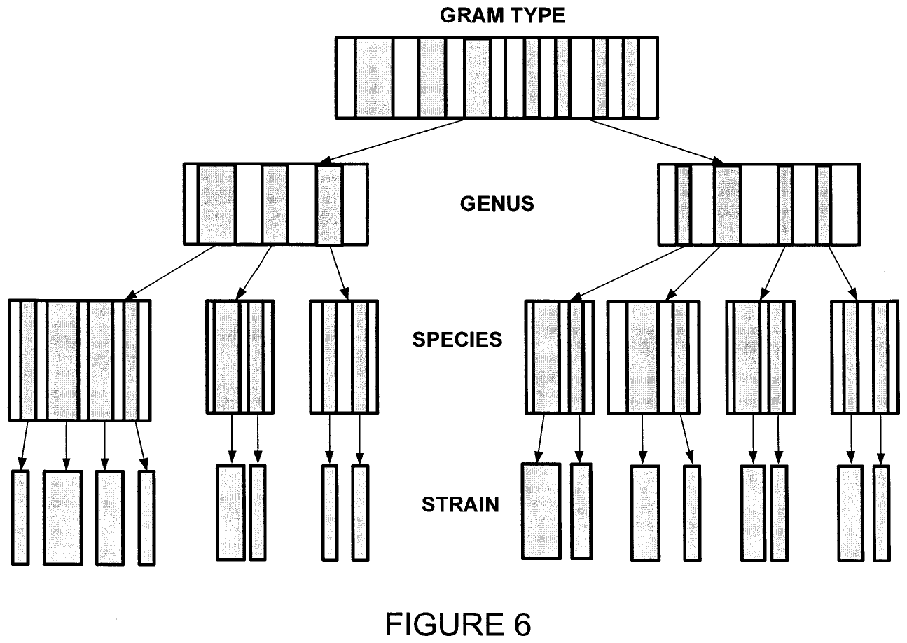

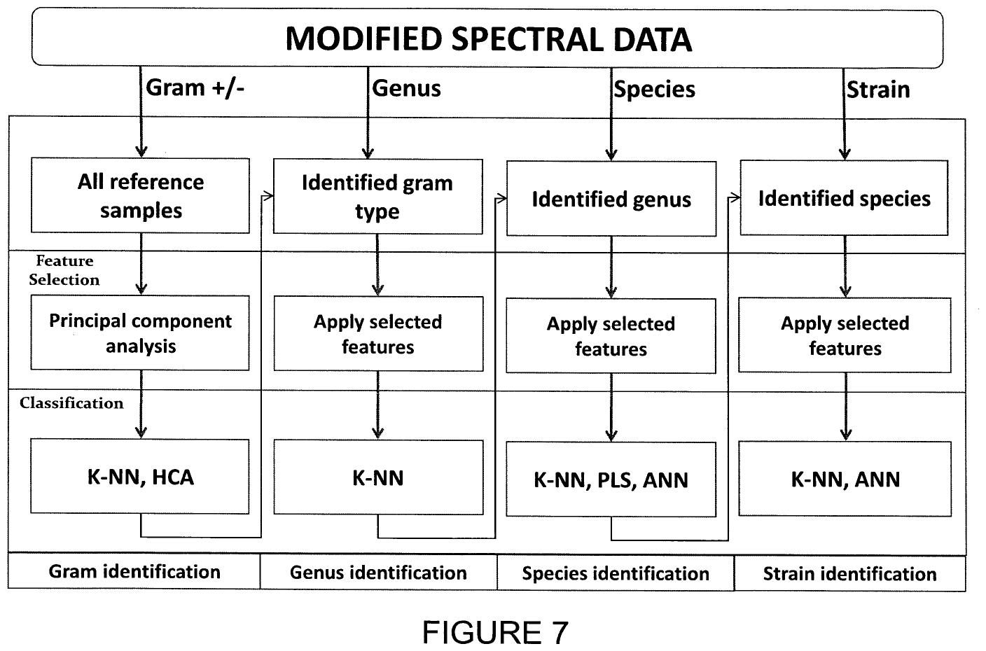

As illustrated in FIG. 6, a multi-tier classification strategy may be used, and classification may be performed at each taxonomic level. Classification models may be developed using appropriate subsets of the spectra in the database as training sets. Each classification model may be optimized using a feature selection algorithm to identify the spectral features that best characterize the desired classification. The shaded regions in FIG. 6 denote selected regions for the feature selection algorithm. The microorganism may thus be classified in accordance with Gram-stain type (i.e. positive or negative), genus, species, and strain, as per FIG. 7. A microorganism may further be determined to be an antibiotic-resistant strain or an antibiotic-sensitive strain. For each level of classification, analysis may be employed to find spectral features that differentiate between types. For example, specific spectral regions within the reflection-FTIR spectrum may be selected to separate the Gram-positive from the Gram-negative bacteria, this followed by a tier-wise separation at the genus, species, strain, and serotype levels and in some cases separation between antibiotic-resistant and antibiotic-sensitive strains and in some cases separation between genotypes. In some cases, toxin-producing bacteria can further be classified by the type of toxin they produce.

The signal-to-noise ratio (SNR) of the spectral data may be improved by performing a greater number of scans of the sample, such as 64, 128, or 256 instead of 4, 16, or 32. However, a greater number of scans means a longer scan time, increasing the difference between the water vapor level in the background spectrum and the spectral data. The method 200 is thus a compromise: obtaining an acceptable SNR while minimizing the difference in water vapor level between the background spectrum and the spectral data. In some embodiments, the selected number of scans for the acquisition of the spectral data is 128. Other numbers of scans may also be used. Spectra acquired from lower number of scans can be co-added to improve the SNR.

In some embodiments, the data selected for analysis from the modified spectral data is taken from a range of about 1480 cm⁻¹ to about 800 cm⁻¹. In some embodiments, the range is about 3030 cm⁻¹ to about 2800 cm⁻¹. In some embodiments, the range is about 1770 cm⁻¹ to about 650 cm⁻¹. Other ranges may also be used.

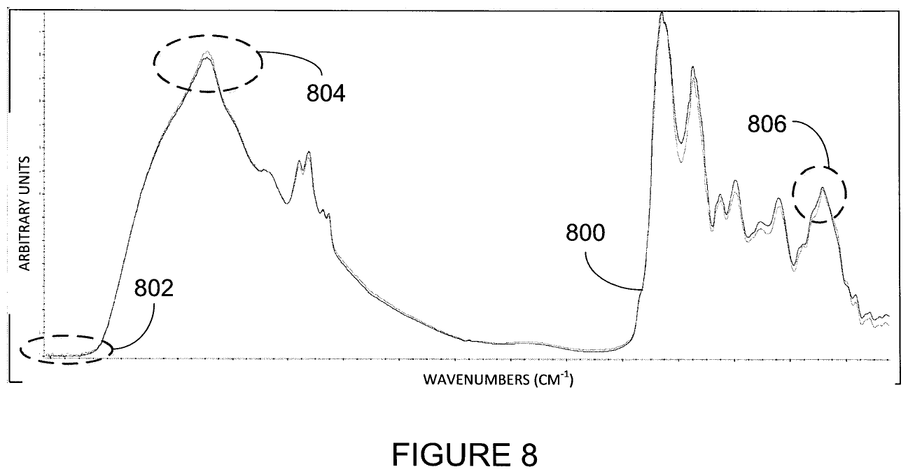

In some embodiments, the modified spectral data is validated with regards to various parameters, such as water vapor level, water content of the sample, and/or biomass of the sample. FIG. 8 is an example of the modified spectral data 800. Region 802 is used to validate water vapor level, region 804 is used to validate sample water content, and region 806 is used to validate biomass. The measurements obtained in each one of the regions 802, 804, 806 may be compared with one or more corresponding threshold and/or range in order to validate each one of the parameters.

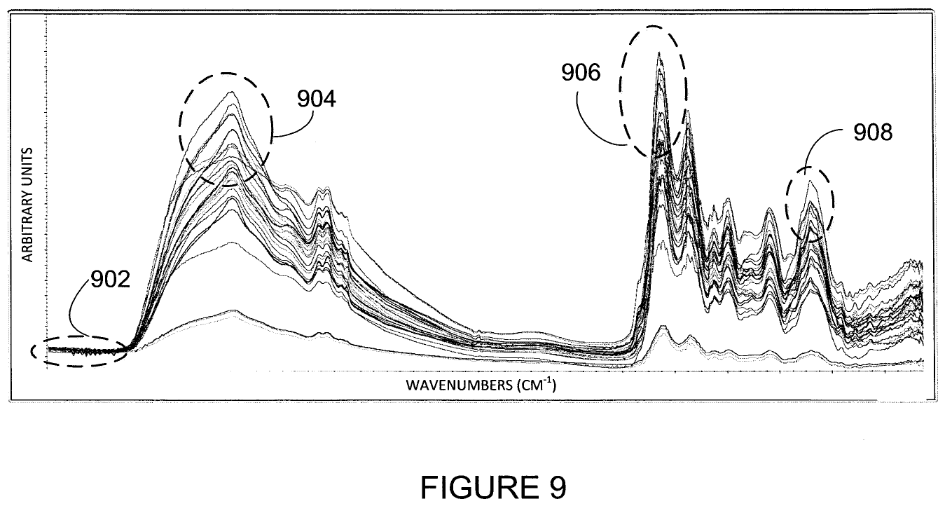

FIG. 9 shows an example of a set of modified spectral data that is non-compliant with regards to water vapor level, as shown in region 902. Therefore, there is spectral interference from water vapor in the modified spectral data. Validation may be performed visually by comparing the captured signal to another signal deemed compliant, or it may be performed automatically by comparing the measured values to a first threshold value.

In another example, the measurements of region 804 are compared to a second threshold. A measurement for water content of the sample is considered compliant if it is above the second threshold, so as to ensure that the water content of the sample is retained at the time of spectral acquisition. In embodiments in which the reflective substrate is indium-tin-oxide coated substrate or steel, an example value for the second threshold is signal intensity in region 804 of 0.8 absorbance units ±0.4 absorbance units. Measurements below the second threshold are indicative of a sample that is too thin (<0.2 absorbance units) or too thick (>1.5 absorbance units). The modified spectral data may be rejected as being non-compliant in such a case. Region 904 in FIG. 9 shows an example of a set of modified spectral data that is also non-compliant with regards to the water content of the sample. Validation may be performed visually by comparing the captured signal to another signal or it may be performed automatically by comparing the measured values to the second threshold value.

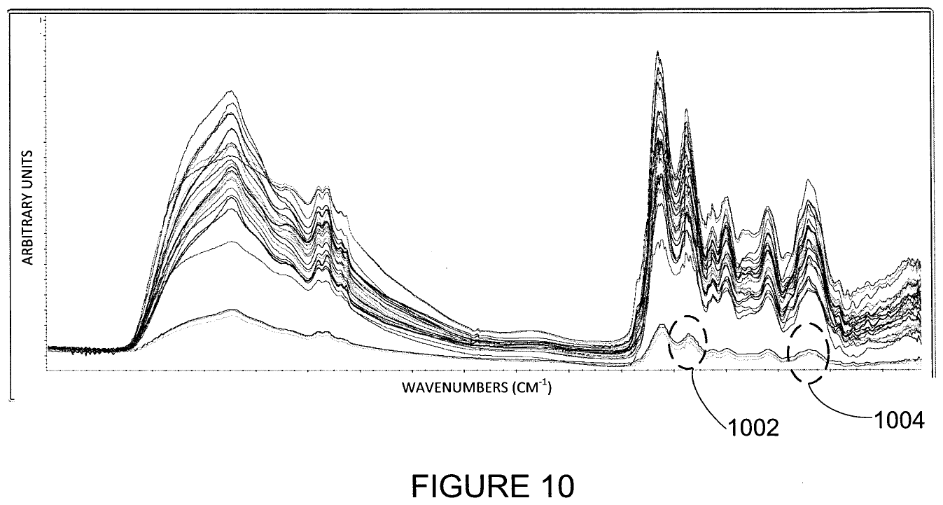

In some embodiments, biomass of the sample is validated in a similar manner as that shown with respect to water vapor level and sample water content. For example, in FIG. 10, regions 1002 and 1004 show a signal lower than a reference signal, which represents a third threshold for biomass. This is indicative of a biomass that is too low and therefore cause for rejection of the spectral data.

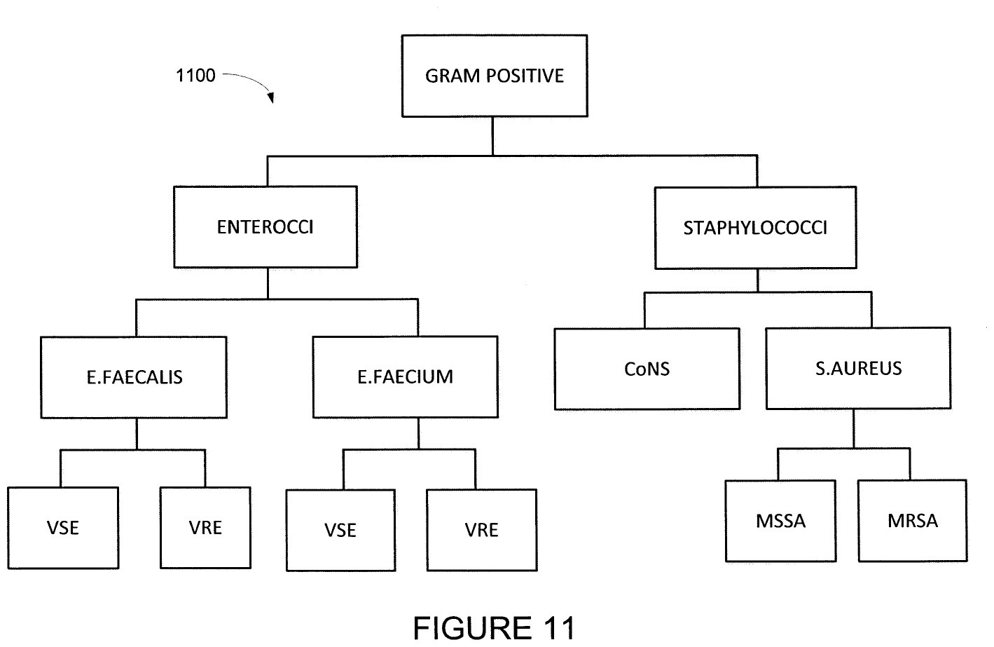

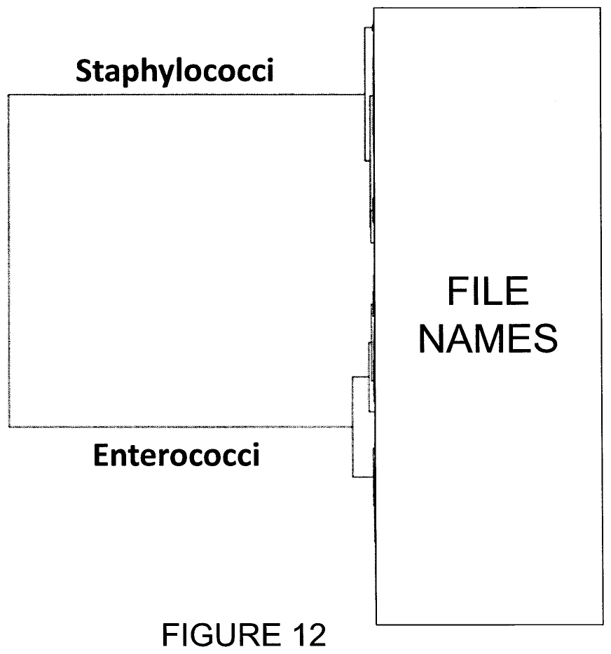

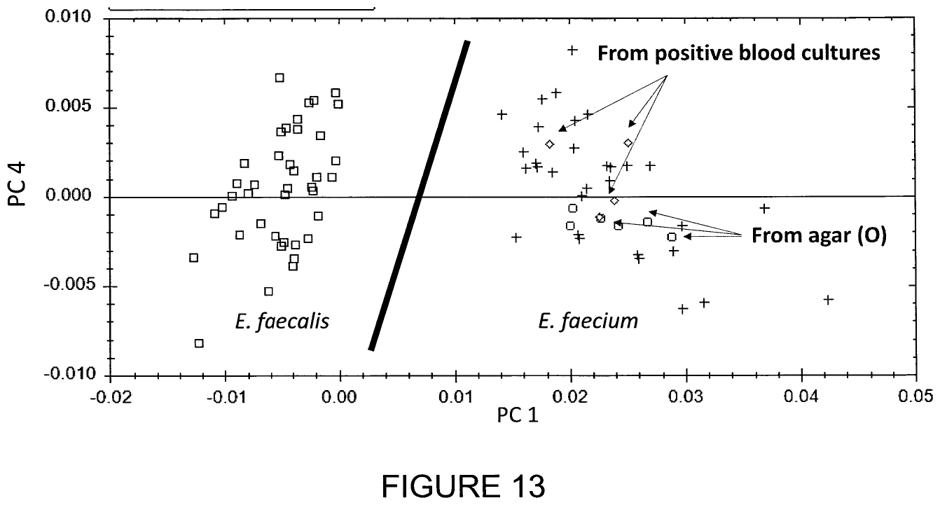

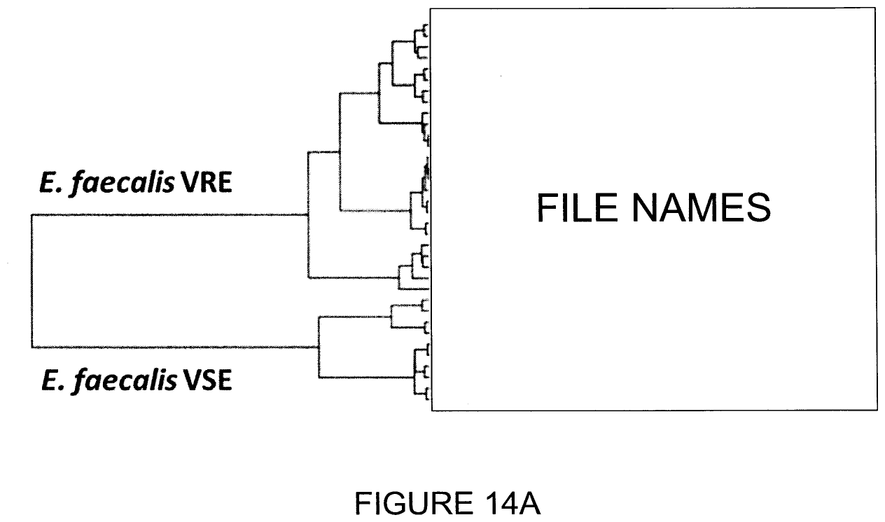

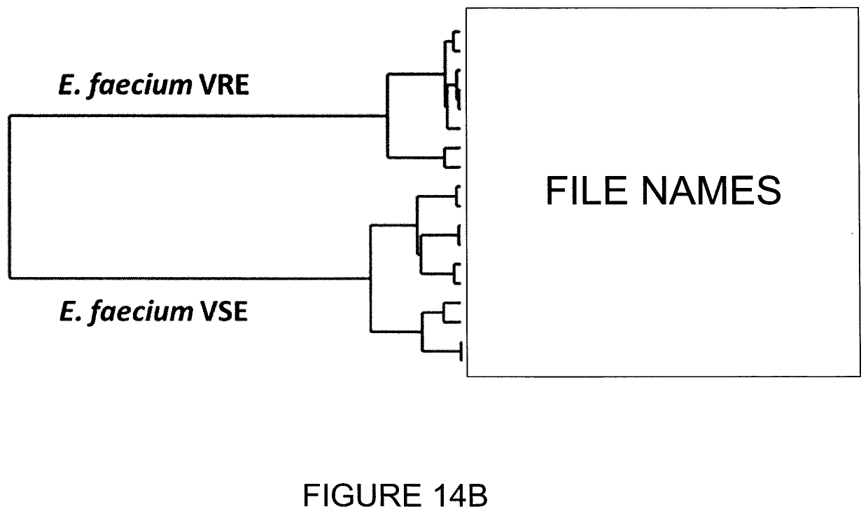

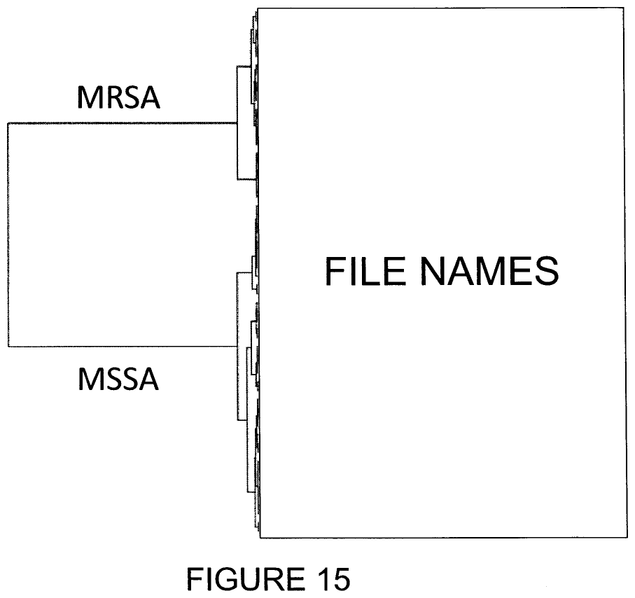

The method 200 may be used to discriminate between Gram-positive and Gram-negative bacteria by principal component analysis (PCA) of reflection-FTIR spectra. An example database structure 1100 for Gram-positive bacteria is illustrated in FIG. 11. The top tier of the structure 1100 represents the Gram-positive bacteria in the spectral database, followed by a second tier to discriminate between enterococci and staphylococci (see FIG. 12). A third tier allows to discriminate between Enterococcus faecalis and E. faecium (see FIG. 13). In the fourth tier, the method 200 may be used to discriminate between vancomycin-resistant enterococci (VRE) and vancomycin-sensitive enterococci (VSE) (see FIGS. 14A and 14B), and to discriminate between methicillin-resistant Staphylococcus aureus (MRSA) and methicillin-sensitive Staphylococcus aureus (MSSA) (see FIG. 15).

The method 200 may be used to identify antibiotic-resistant strains of microorganisms taken from culture media without the addition of any antibiotic. FIGS. 14A and 14B illustrate HCA plots showing clustering of clinical isolates of vancomycin-resistant enterococci (VRE) and vancomycin-sensitive enterococci (VSE) based on differences in their reflection-FTIR spectra following culture on 5% sheep's blood agar.

In the creation of a spectral database, the microorganisms may be cultured twice to ensure purity. Isolated colonies with the same morphology are selected and transferred to the surface of the infrared reflective substrate for reflection-FTIR spectroscopic measurement. The reflection-FTIR spectrum is recorded. Replicate spectra may be obtained and those with the smallest standard deviation from the mean, are added to the database. Additional information may be added to a spectral file header, such as genus, species, strain, antimicrobial profile, growth medium, growth conditions, date, and the like.

In some embodiments, the modified spectral data is compared with spectral data of reference microorganisms obtained using a same culture medium as the sample. The use of another culture medium may result in an altered spectral profile. Therefore, the same media may be used to ensure that the same spectral profile is obtained. Alternatively, spectral data of reference microorganisms are obtained using a plurality of different culture media, and data from each spectral acquisition are pooled in order to make the reference data culture-media independent.

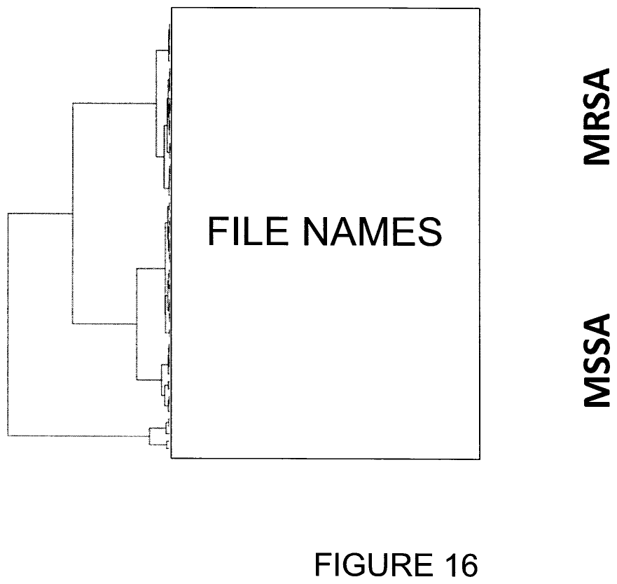

The method 200 may be used to identify microorganisms from positive blood cultures. While traces of blood in dried samples act as large contaminants, having the blood diluted in water causes the effect to be negligible. FIG. 16 illustrates a dendrogram plot showing clustering of methicillin-resistant Staphylococcus aureus (MRSA) and methicillin-susceptible Staphylococcus aureus (MSSA) clinical isolates based on differences in their reflection-FTIR spectra.

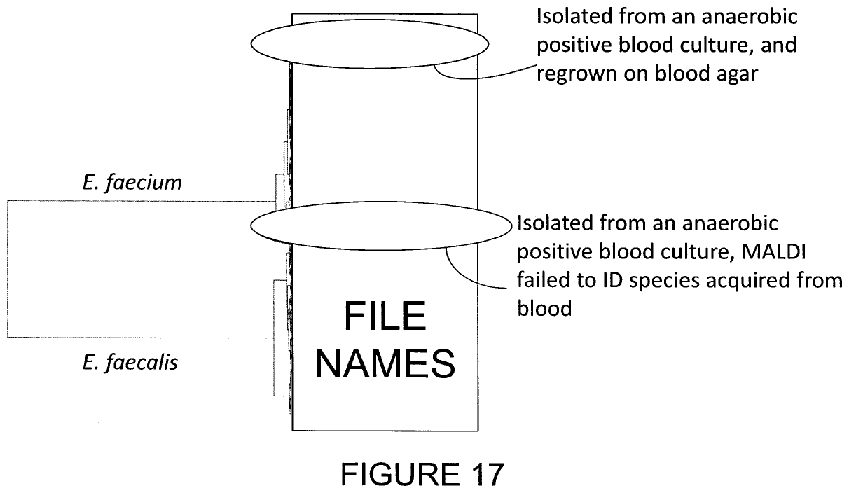

In some embodiments, infrared spectroscopy as described herein is used to enhance and/or refine characterization of a microorganism by MALDI-TOF MS. Referring to FIG. 17, there is illustrated an example of differentiation between Enterococcus faecalis and E. faecium with the use of an FSA and the identification of E. faecium isolated from positive blood culture and confirmed by conventional analysis. MALDI-TOF MS analysis did not provide the correct identification, presumably due to trace blood or extraneous proteins from the microbial extract.

In some embodiments, the m/z data is combined with the reflection-FTIR data into a single stitched spectrum and an FSA is used to identify the mass spectral and infrared spectral features that maximize the differentiation between two types of microorganisms.

In some embodiments, the prediction of the identity of an unknown microorganism is carried out by reflection-FTIR spectral analysis independent to the MALDI-TOF MS analysis. The identification of the unknown microorganism by the two independent means can further enhance the reliability of the identification by MALDI-TOF MS.

In some embodiments, other spectral data is acquired from another spectroscopic technique-such as ¹H (proton), ¹³C, ³¹P or ¹⁵N nuclear magnetic resonance (NMR) spectroscopy, including solid-state high-resolution magic angle spinning (HRMAS) NMR. The reflection-FTIR data may thus be used to identify the spectral features responsible for the differentiation between two types of microorganisms. Subsequently or in tandem, other spectral data from other spectroscopic techniques can be utilized to identify the biomarker(s) associated with the infrared spectral features. In some embodiments, spectra generated from stitching of multiple spectral data sets from the above-mentioned techniques can be subjected to analysis with the use of an FSA after spectral pre-processing, including normalization. Individually or combined, these pre-processing methods increase the reliability of microbial identification by multispectral domain spectroscopy.

An example protocol for the separation of bacteria from blood culture broth for the purpose of identification by reflection FTIR spectroscopy is as follows. Specific and non-limiting values are provided for illustrative purposes only.

1. Aliquots from positive blood cultures are syringed into BD Vacutainer® SST™ serum separator tubes.
2. Tubes are centrifuged at 3,000 rpm for 10 minutes.
3. The supernatant is removed and replaced with equal volume of saline.
4. Tubes are centrifuged at 3,000 rpm for 10 minutes.
5. The supernatant is removed.
6. Residual supernatant is removed by using a cotton swab and bacteria are transferred to the surface of the infrared reflective substrate by using a plastic applicator.

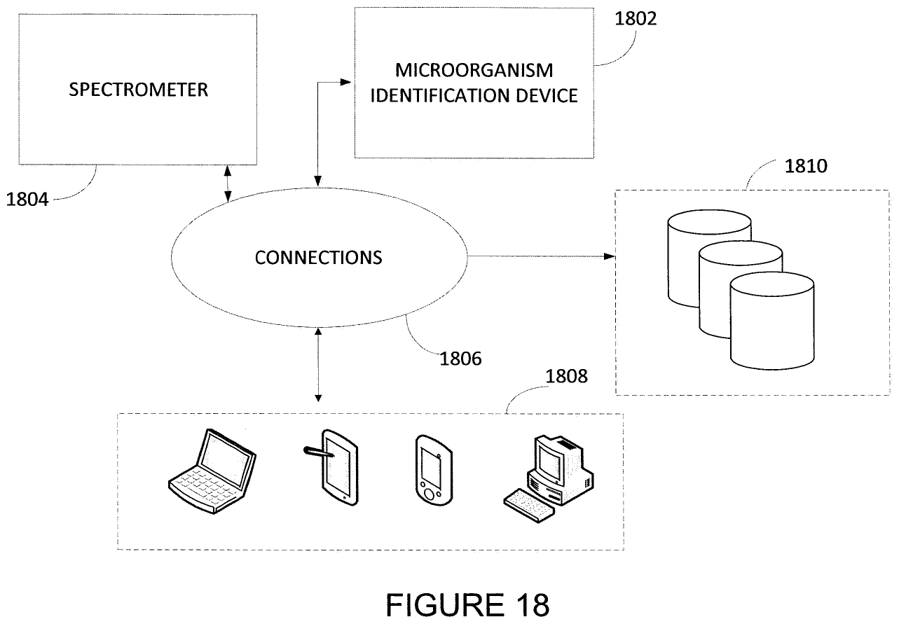

It should be noted that the sample may have been previously treated using various processes, such as those associated with clinical samples, subcultures, and/or frozen samples. For example, immuno-capture methods for extraction of microorganism from blood (or other bodily fluids) employing magnetic beads form a bacteria-bead complex that can be directly measured by reflection-FTIR spectroscopy. Referring to FIGS. 18 to 20, a system for spectral identification of microorganisms will now be described. In FIG. 18, there is illustrated a microorganism identification device 1802 operatively connected to spectrometer 1804. The microorganism identification device 1802 may be provided separately from or incorporated within the spectrometer 1804. For example, the microorganism identification device 1802 may be integrated with the spectrometer 1804, either as a downloaded software application, a firmware application, or a combination thereof. The spectrometer 1804 may be any instrument capable of acquiring infrared spectral data from an object, such as but not limited to an FTIR spectrometer. Some example spectral acquisition parameters are as follows:

Resolution: 8 cm⁻¹
Zero filling: 0-8 orders
Detector type: DTGS or MCT or FPA
Detector gain: 1-4
Apodization: triangular or Happ-Ganzel
Number of scans: 8-256
Time of acquisition: 10-300 seconds
Background (before each sample_: 16-256 scans)
SNR: >1,000:1 (or 1 mAu between 1380 and 980 cm⁻¹) (100% line, 64 co-added scans/8 cm⁻¹ resolution) with residual water vapor <0.005 Au

In some embodiments, the following protocol may be used for acquiring the background spectrum and spectral data with the spectrometer 1804.

1. Turning on the instrument and letting it warm up.
2. Launching the software on the computer and setting the spectral acquisition parameters to:
Number of scans: 128 scans (or another value, as desired)
Resolution: 4-8 cm⁻¹
   3. Collecting a background spectrum (noting that the surface of the infrared-reflective substrate must be bare, clean & dry).
   4. Collecting a small amount of bacteria (˜1-5 colonies) from a culture plate using a sterilized toothpick or loop without breaking the culture medium surface.
   5. Spreading the collected bacteria on the surface of the infrared reflective substrate or MALDI-TOF MS slide (˜2-3 mm in diameter).
   6. Pressing “Scan sample” to collect the spectral data.
   7. Discarding or cleaning the infrared reflective surface by wetting the bacteria with a disinfecting fluid (70% ethanol or bleach).
   8. Wiping the bacteria off using a Kimwipe.
   9. Repeating steps 3 through 8 for each subsequent sample and acquiring a spectrum of a preselected reference strain after every 30 samples. These numbers are purely illustrative and may be varied.
   10. Cleaning the surface of the infrared reflective substrate by the procedure in step 8 (or discarding the infrared reflective substrate) and turning off the instrument.

The following experimental protocol was used for reflection-FTIR spectral acquisition. Gram-positive isolates were sub-cultured on 5% sheep's blood agar for 18-24 h at 35° C. With certain exceptions, Gram-negative isolates were sub-cultured on 5% sheep's blood agar or MacConkey agar for 18-24 h at 35° C. Following incubation, 1-5 isolated colonies were collected from the agar surface and spread on the surface of the infrared reflective substrate of the FTIR spectrometer and a spectrum was immediately recorded using a spectral acquisition time of 45 seconds. For each culture plate, 2-3 replicate spectra were acquired from different colonies.

Referring back to FIG. 18, various types of connections 1806 may be provided to allow the microorganism identification device 1802 to communicate with the spectrometer 1804. For example, the connections 1806 may comprise wire-based technology, such as electrical wires or cables, and/or optical fibers. The connections 1806 may also be wireless, such as RF, infrared, Wi-Fi, Bluetooth, and others. Connections 1806 may therefore comprise a network, such as the Internet, the Public Switch Telephone Network (PSTN), a cellular network, or others known to those skilled in the art. Communication over the network may occur using any known communication protocols that enable devices within a computer network to exchange information. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol), and Ethernet. The connections 1806 may also use various encryption means to protect any of the data acquired and/or transferred.

The microorganism identification device 1802 may be accessible remotely from any one of a plurality of devices 1808 over connections 1806. The devices 1808 may comprise any device, such as a personal computer, a tablet, a smart phone, or the like, which is configured to communicate over the connections 1806. In some embodiments, the microorganism identification device 1802 may itself be provided directly on one of the devices 1808, either as a downloaded software application, a firmware application, or a combination thereof.

One or more databases 1810 may be integrated directly into the microorganism identification device 1802 or any one of the devices 1808, or may be provided separately therefrom (as illustrated). In the case of a remote access to the databases 1810, access may occur via connections 1806 taking the form of any type of network, as indicated above. The various databases 1810 described herein may be provided as collections of data or information organized for rapid search and retrieval by a computer. The databases 1810 may be structured to facilitate storage, retrieval, modification, and deletion of data in conjunction with various data-processing operations. The databases 1810 may be any organization of data on a data storage medium, such as one or more servers or long-term data storage devices. The databases 1810 illustratively have stored therein spectral data for reference microorganisms used for comparison with spectral data of unknown samples.

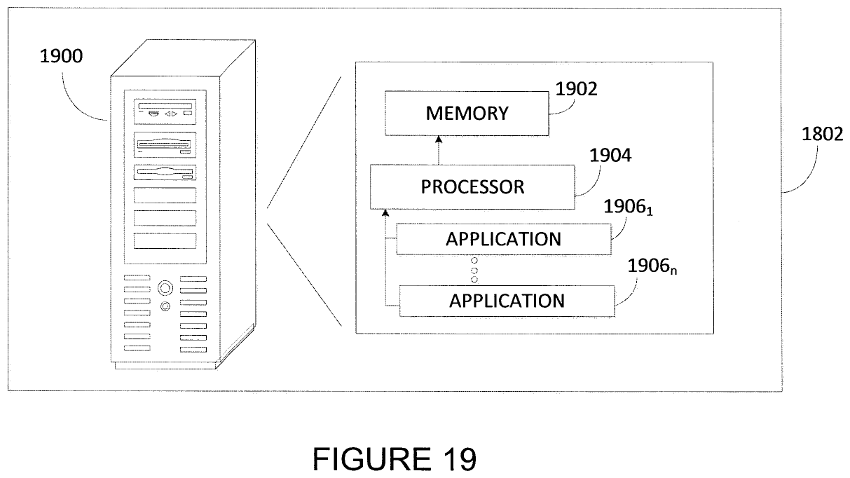

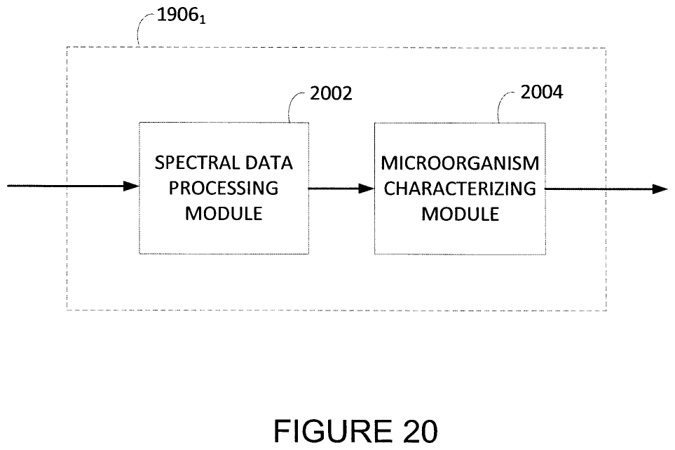

As shown in FIG. 19, the microorganism identification device 1802 illustratively comprises one or more servers 1900. For example, a series of servers corresponding to a web server, an application server, and a database server may be used. These servers are all represented by server 1900 in FIG. 20. The server 1900 may be accessed by a user, such as a technician or laboratory worker, using one of the devices 1808, or directly on the system 1802 via a graphical user interface. The server 1900 may comprise, amongst other things, a plurality of applications 1906₁ . . . 1906ₙ running on a processor 1904 coupled to a memory 1902. It should be understood that while the applications 1906₁ . . . 1906ₙ presented herein are illustrated and described as separate entities, they may be combined or separated in a variety of ways.

The memory 1902 accessible by the processor 1904 may receive and store data. The memory 1902 may be a main memory, such as a high-speed Random Access Memory (RAM), or an auxiliary storage unit, such as a hard disk, a floppy disk, or a magnetic tape drive. The memory 1902 may be any other type of memory, such as a Read-Only Memory (ROM), or optical storage media such as a videodisc and a compact disc. The processor 1104 may access the memory 1902 to retrieve data. The processor 1904 may be any device that can perform operations on data. Examples are a central processing unit (CPU), a front-end processor, a microprocessor, and a network processor. The applications 1906₁ . . . 1906ₙ are coupled to the processor 1904 and configured to perform various tasks. An output may be transmitted to the devices 1808.

FIG. 20 is an exemplary embodiment of an application 1906₁ running on the processor 1904. The application 1906₁ illustratively comprises a spectral data processing module 2002 and a microorganism characterizing module 2004. The spectral data processing module 2002 is configured for receiving the background spectrum and the spectral data. The spectral data processing module 2002 may also be configured for combining the background spectrum and the spectral data to produce the modified spectral data. In some embodiments, the spectral data processing module is further configured for validating the modified spectral data, for example by comparing water vapor level, sample water content, and/or sample biomass to a threshold or a reference value. Some of the mathematical operations performed by the spectral data processing module 2002 on the background spectrum and/or spectral data include, but are not limited to, first derivatives, vector normalizations (4000-400 cm⁻¹), and cubic interpolation (with data spacing of 0.1-32).

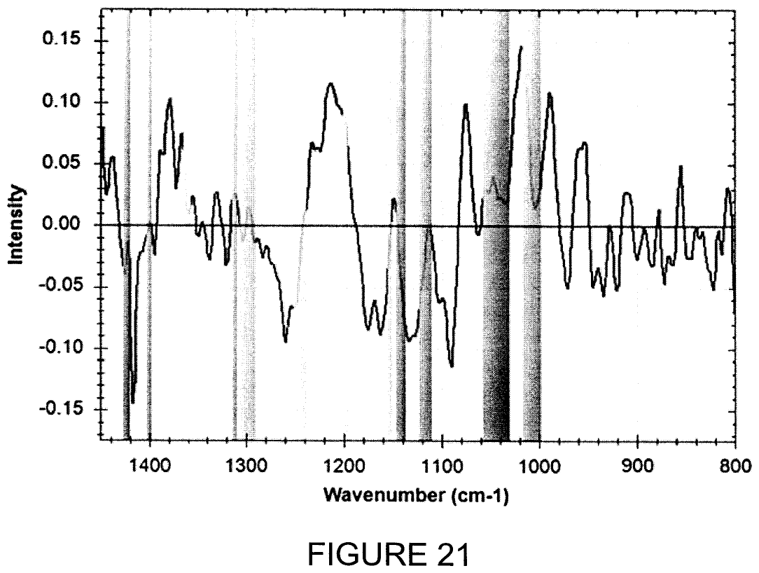

The microorganism characterizing module 2004 may be configured to receive the modified spectral data and to perform microorganism characterization by comparing the modified spectral data to reference spectral data of known microorganisms. In some embodiments, the microorganism characterizing module 2004 is configured to use target spectral regions in the modified spectral data pre-selected by applying a feature selection algorithm to training data as per U.S. Pat. No. 9,551,654. For example, an FSA is employed to identify the significant biochemical markers that are more relevant than the proteins in microbial identification. The comprehensive information content in the reflection-FTIR spectra can differentiate between types of bacteria at different levels of classification (genus, species, strain, serotype, and antimicrobial resistance characteristics and in some cases genotypic characteristics). Based on the FSA, spectral regions attributed to specific class of biomolecules (example, polysaccharides, lipids, proteins or nucleic acids) can then be identified to increase the resolution power of MALDI-TOF MS in its ability to differentiate between closely related genera, such as E. coli and Shigella. FIG. 21 illustrates an example of results obtained by applying the FSA.

In some embodiments, a grid-greedy feature selection algorithm is used with three regions of a minimum size of 20 wavenumbers (6 features) and a maximum size of 92 wavenumbers (24 features) per region. All possible combinations of such regions are evaluated between 3050 and 2700 cm⁻¹ and between 1780 and 400 cm⁻¹ and the region with the highest LOOCV-KNN classification score is selected. The greedy portion of the algorithm examines combinations of adjacent features following the path of greatest improvement. The forward selection begins by evaluating the single feature with the highest classification score, followed by adding features one at a time which keeps the score at a maximum. The routine stops when the classification score is no longer improved by adding features. The search may continue for a minimum of 6 features (1 of the total number of features) even if there is no further improvement in classification score in order to minimize over-fitting of the training data. Other feature selection algorithms may also be used.

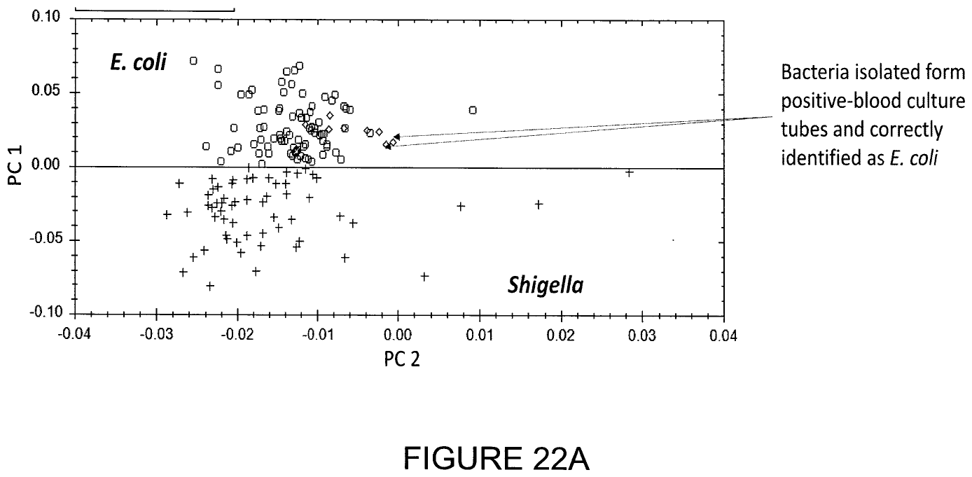

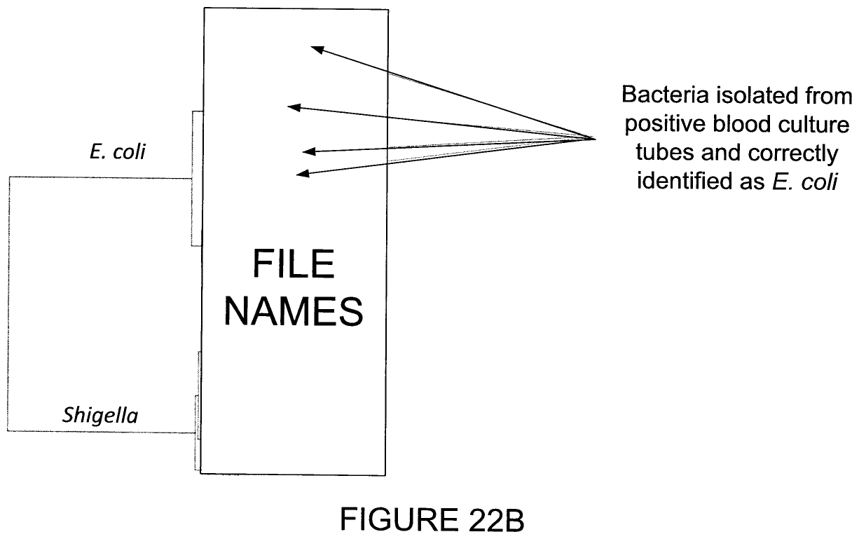

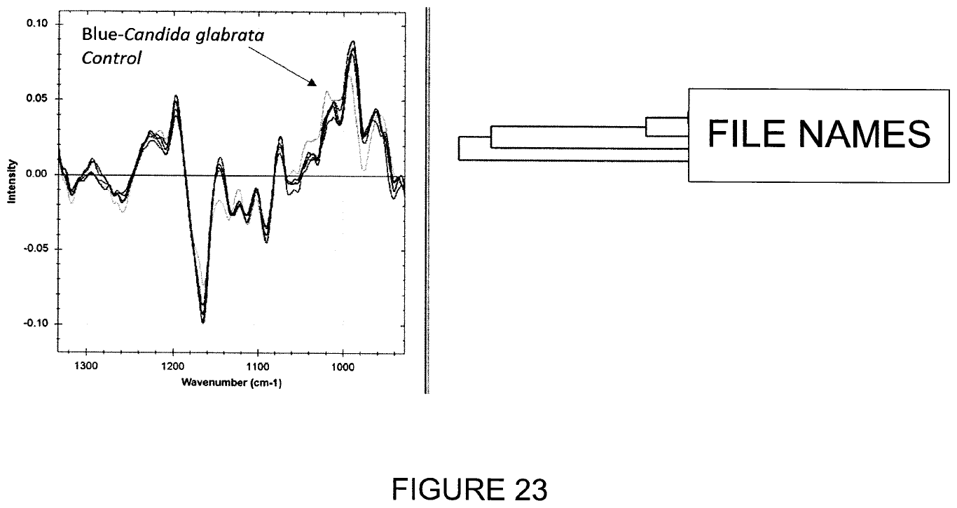

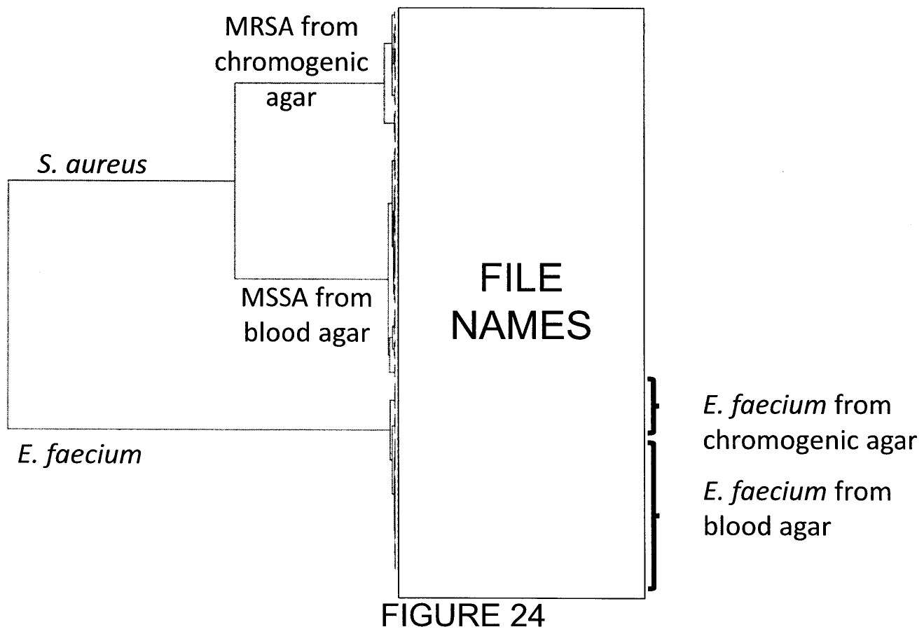

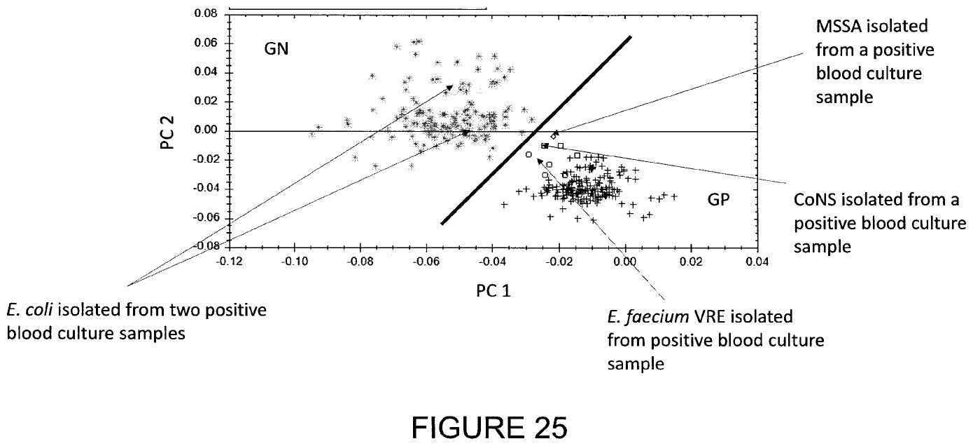

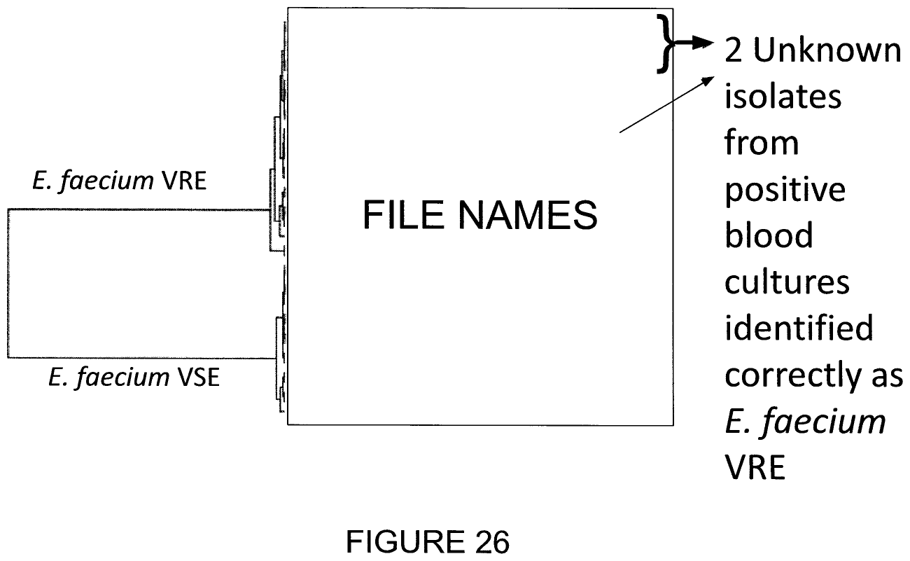

Additional examples are provided in FIGS. 22 to 31. FIGS. 22A and 22B illustrate the differentiation between E. coli and Shigella species and the identification of bacteria isolated from positive blood culture tubes as E. coli based on reflection-FTIR spectral data in the spectral range of 899-904 cm⁻¹. FIG. 23 is an example of identification of a yeast clinical isolate based on reflection-FTIR spectral data in the spectral range of 1520-980 cm⁻¹. FIG. 24 is an example of a dendrogram obtained by HCA of reflection-FTIR spectra of antibiotic-sensitive and antibiotic-resistant microorganisms. FIG. 25 is an example of differentiation between Gram-positive and Gram-negative microorganisms, including microorganisms extracted directly from positive blood cultures, by HCA of reflection-FTIR spectral data in the spectral ranges of 832-835, 944-947, 1285-1288, 1302-1305, 1430-1432, and 2859-2861 cm⁻¹. FIG. 26 is an example of the correct classification of clinical isolates of vancomycin-resistant E. faecium based on the comparison of their reflection-FTIR spectra with reflection-FTIR spectra in a spectral database.

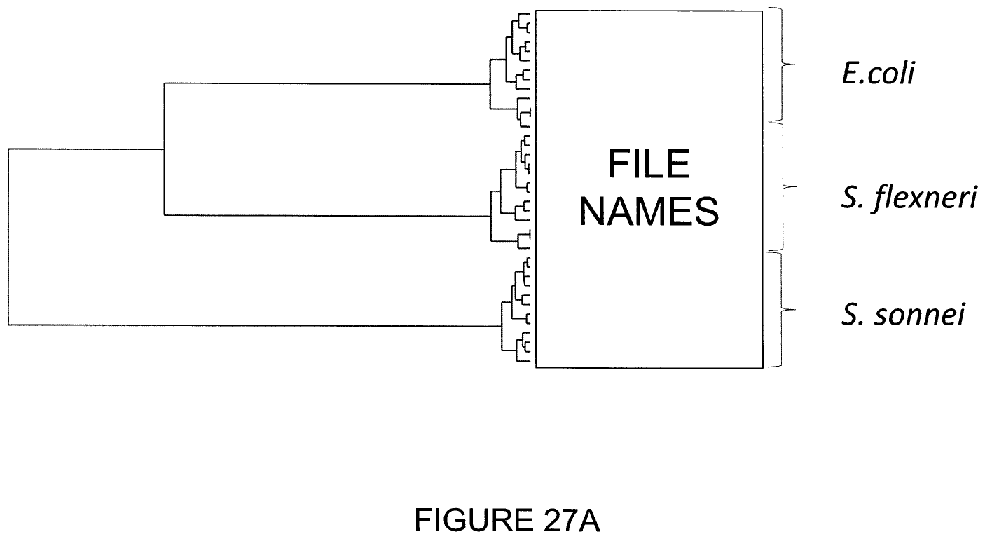

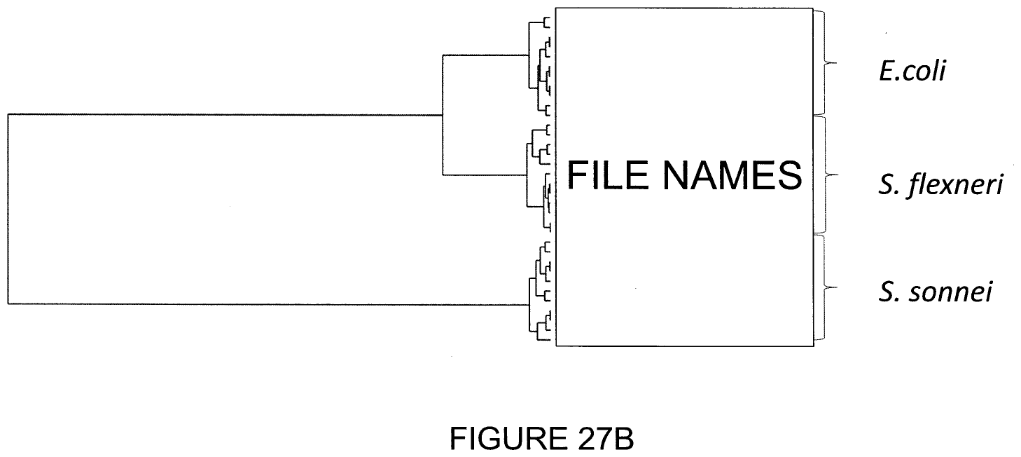

FIG. 27A is a dendrogram obtained by HCA of reflection-FTIR spectra acquired from microorganisms on MALDI-TOF MS slides. In this example, the spectral ranges of 1169-1179, 1316-1324, 1339-1352, 2801-2808, 2852-2860, and 3002-3011 cm⁻¹ were employed for the differentiation among E. coli, Shigella sonnei and Shigella flexneri. Note that MALDI-TOF MS is not capable of discriminating between E. coli and Shigella species. FIG. 27B is another example of results obtained from reflection-FTIR measurements performed on MALDI-TOF MS slides; in this example, the microorganisms were inactivated by exposure to aqueous ethanol (70%) prior to spectral acquisition. Spectral ranges of 1040-1044, 1052-1056, and 1172-1176 cm⁻¹ were employed for the differentiation among E. coli, Shigella sonnei and Shigella flexneri.

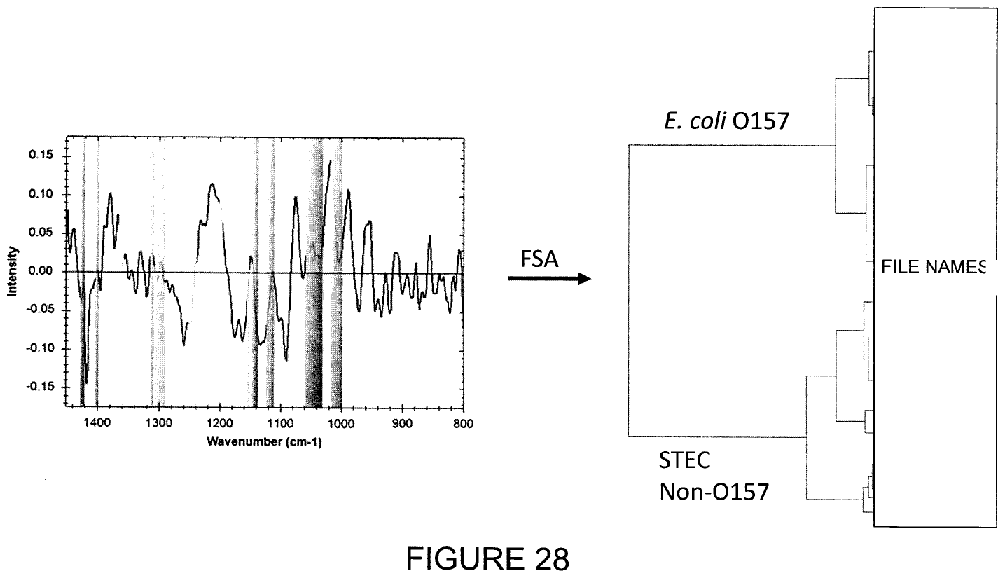

FIG. 28 illustrates an example of a first-derivative reflection-FTIR spectrum of Shiga toxin-producing E. coli (STEC) microorganisms recorded by reflection-FPA-FTIR spectroscopy from an isolate deposited on a MALDI-TOF MS slide and the dendrogram generated by HCA differentiating E. coli O157 from other serotypes of STEC. The FSA identified spectral ranges of 999-1018, 1031-1059, 1111-1124, 1138-1148, 1152-1157, 1196-1202, 1239-1252, 1291-1308, 1310-1315, 1358-1367, 1399-1404, and 1421-1429 cm⁻¹ for achieving differentiation.

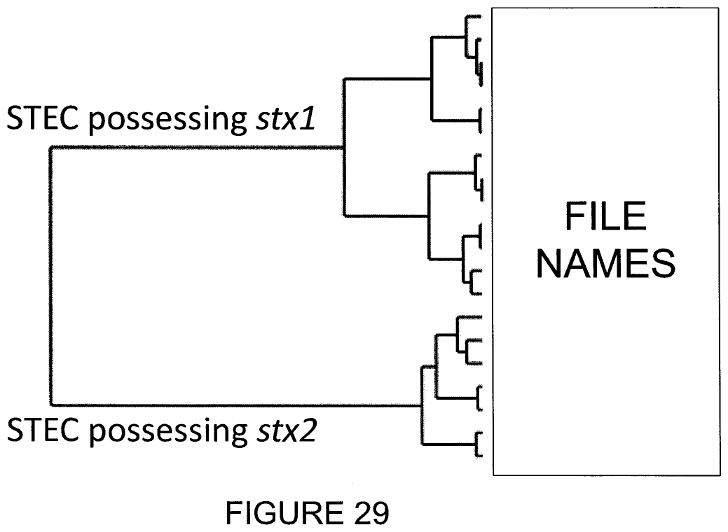

FIG. 29 illustrates an example of differentiation of STEC microorganisms by HCA of spectra recorded by reflection-FPA-FTIR spectroscopy from isolates deposited on a MALDI-TOF MS slide. Spectral ranges of 957-968, 1107-1119, 1142-1157, 1169-1180, 1281-1296, 1319-1335, and 3008-3024 cm⁻¹ were identified for the differentiation between STEC isolates possessing genes encoding different toxin types.

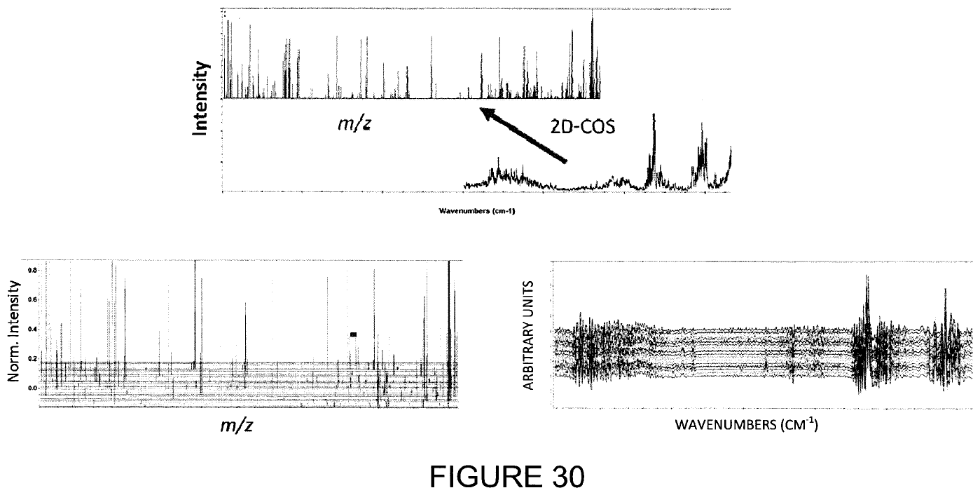

FIG. 30 illustrates the correlation of spectral features in the reflection-FTIR spectra to m/z lines derived from MALDI-TOF MS for the purpose of identifying m/z values for the discrimination among closely related microorganisms.

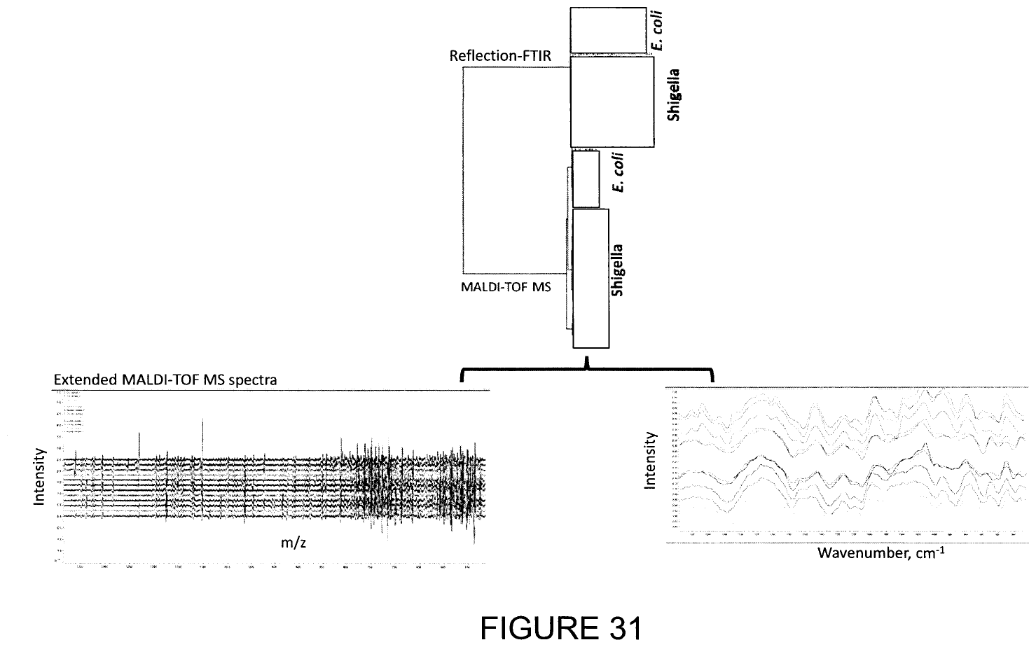

FIG. 31 illustrates an example of reflection-FTIR spectra coupled with MALDI-TOF MS for enhancing the discrimination between E. coli and Shigella, using the spectral regions of 1169-1179, 1316-1324, 1339-1352, 2801-2808, 2852-2860, 3003-3011 cm⁻¹, 746-748, 817-819, 934-935, 1097-1098, 1193-1195, 1225-1227, 1234-1236, 1274-1276, 1343-1345 Da (m/z).

The methods and systems described herein employ a simple and universally applicable protocol that requires minimal sample preparation and no reagent beyond a culturing step. The methods may be used with a high degree of automation and is amenable to micro-colony analysis. They produce a fast turnaround time at a low cost per test, and are capable of detecting biochemical differences between antibiotic-resistant and antibiotic-sensitive bacterial strains in the absence or in the presence of the antibiotic.

The methods and systems described herein may also be used for recording spectra from microorganisms grown on agar in the vicinity of antibiotic-impregnated discs. Alternatively, the antibiotics can be incorporated into the agar matrix (in the presence or absence of a chromogenic agent). Direct identification of bacteria may be performed using reflection-FTIR spectroscopy as described herein.

The methods and systems described herein may also be used for the identification of clinical isolates from positive blood cultures. Indeed, as long as there is sufficient microorganism biomass that can be obtained from a positive blood culture (in the presence or absence of selective antibiotics), direct identification of bacteria may be performed using reflection-FTIR spectroscopy as described herein.

In some embodiments, the reflection-FTIR spectroscopic methods and systems described herein can be complemented by MALDI-TOF MS and/or HRMAS NMR (high-resolution magic-angle spinning NMR), for example, for the discrimination between MRSA and MSSA, VRE and VSE, and E. coli and Shigella spp. The methods and systems may also be used for the identification of Shiga-toxin-producing E. coli (STEC).

In some embodiments, portable reflection-FTIR spectrometers may be used to perform the methods and implement the systems described herein.

It has been demonstrated that reflection-FTIR and reflection-FPA-FTIR spectra recorded from bacteria deposited on MALDI-TOF MS slides can compensate for the limitations of MALDI-TOF MS, such as the inability to discriminate between E. coli and Shigella. It has also been demonstrated that microbiology laboratories can effectively employ their current MALDI-TOF MS SOP with the methods and systems described herein to overcome MALDI-TOF MS limitations. In particular, MALDI-TOF MS is generally unable to discriminate between antibiotic-sensitive and antibiotic-resistant bacteria. The techniques and methods described herein may be used to discriminate between antibiotic-sensitive and antibiotic-resistant bacteria.

The above description is meant to be exemplary only, and one skilled in the relevant arts will recognize that changes may be made to the embodiments described without departing from the scope of the invention disclosed. For example, the blocks and/or operations in the flowcharts and drawings described herein are for purposes of example only. There may be many variations to these blocks and/or operations without departing from the teachings of the present disclosure. For instance, the blocks may be performed in a differing order, or blocks may be added, deleted, or modified. While illustrated in the block diagrams as groups of discrete components communicating with each other via distinct data signal connections, it will be understood by those skilled in the art that the present embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present embodiment. The present disclosure may be embodied in other specific forms without departing from the subject matter of the claims. Also, one skilled in the relevant arts will appreciate that while the systems, methods and computer readable mediums disclosed and shown herein may comprise a specific number of elements/components, the systems, methods and computer readable mediums may be modified to include additional or fewer of such elements/components. The present disclosure is also intended to cover and embrace all suitable changes in technology. Modifications which fall within the scope of the present invention will be apparent to those skilled in the art, in light of a review of this disclosure, and such modifications are intended to fall within the appended claims.

1. A method for spectral identification of a microorganism, the method comprising:
acquiring a background spectrum to measure a water vapor level of an ambient atmosphere in the absence of a sample;
bringing the sample containing the microorganism into contact with an infrared reflective substrate, the sample having intact microbial cells;
acquiring spectral data from the sample using reflection infrared spectroscopy no more than a predetermined time after having acquired the background spectrum;
combining the background spectrum and the spectral data, thereby producing modified spectral data; and
characterizing the microorganism using the modified spectral data.

2. The method of claim 1, wherein the infrared reflective substrate is a substrate coated with a material having an infrared-reflecting property or comprised of a material having an infrared-reflecting property.

3. (canceled)

4. The method of claim 1, wherein the infrared reflective substrate is at least one of an indium-tin-oxide coated substrate, a metal-coated substrate, a metal oxide-coated substrate, a steel substrate, an E-glass substrate, a metal substrate, a metal oxide substrate, and a matrix-assisted laser desorption/ionization time of flight mass spectrometry slide.

5. (canceled)

6. (canceled)

7. The method of claim 1, wherein the background spectrum is acquired in a path between an infrared source and an infrared detector defined for acquisition of the spectral data while the infrared reflective substrate is without the sample.

8. The method of claim 1, wherein the spectral data is acquired from the sample prior to or after having added a matrix-assisted laser desorption/ionization time of flight mass spectrometry chemical matrix thereto.

9. The method of claim 1, wherein combining the background spectrum and the spectral data comprises computing a logarithm of the spectral data divided by the background spectrum to obtain the modified spectral data.

10. The method of claim 1, further comprising at least one of:
comparing a water vapor level of the modified spectral data to a first threshold and rejecting the modified spectral data when the water vapor level is above the first threshold; and
comparing a water content level of the modified spectral data to a second threshold and rejecting the modified spectral data when the water content level is below the second threshold.

11. (canceled)

12. The method of claim 1, further comprising comparing a biomass of the sample, extracted from the modified spectral data, to a third threshold and rejecting the modified spectral data when the biomass is below the third threshold.

13. The method of claim 1, wherein the sample has a limited free water content and an intact associated and bound water content.

14. The method of claim 1, wherein the microorganism in the sample has a water activity of less than 0.999.

15. The method of claim 1, further comprising applying a vacuum to the sample on the infrared reflective substrate prior to acquiring the spectral data from the sample using reflection infrared spectroscopy.

16. The method of claim 1, wherein acquiring spectral data from the sample comprises at least one of:
acquiring spectral data from the sample in the absence of a drying treatment being applied to the sample;
acquiring spectral data from the sample in the absence of a reagent being applied to the sample for reducing water content of the sample; and
acquiring Fourier transform infrared spectrum.

17. (canceled)

18. (canceled)

19. The method of claim 18, further comprising using the modified spectral data to enhance the characterization of the microorganism by matrix-assisted laser desorption/ionization time of flight mass spectrometry.

20. The method of claim 1, wherein characterizing the microorganism comprises comparing the modified spectral data to reference data to determine an identity of the microorganism.

21. A system for spectral identification of a microorganism, the system comprising:
at least one processing unit; and
a non-transitory computer-readable memory having stored thereon program instructions executable by the at least one processing unit for:
receiving a background spectrum to measure a water vapor level of an ambient atmosphere in the absence of a sample;
receiving spectral data from the sample in contact with an infrared reflective substrate using reflection infrared spectroscopy and acquired no more than a predetermined time after having acquired the background spectrum, the sample containing the microorganism and having intact microbial cells;
combining the background spectrum and the spectral data, thereby producing modified spectral data; and
characterizing the microorganism using the modified spectral data.

22. The system of claim 21, wherein the infrared reflective substrate is any one of an indium-tin-oxide coated substrate, a steel substrate, an E-glass substrate, a metal substrate, a metal-coated substrate, a metal oxide substrate, and a metal oxide-coated substrate.

23. The system of claim 21, wherein the background spectrum is acquired in a path between an infrared source and an infrared detector defined for acquisition of the spectral data while the infrared reflective substrate is without the sample.

24. The system of claim 21, wherein the spectral data is acquired from the sample prior to or after having added a matrix-assisted laser desorption/ionization time of flight mass spectrometry chemical matrix thereto.

25. The system of claim 21, wherein the sample has a limited free water content and an intact associated and bound water content.

26. The system of claim 21, wherein the microorganism in the sample has a water activity of less than 0.999.

27. The system of claim 21, wherein the program instructions are further executable for causing a vacuum to be applied to the sample on the infrared reflective substrate prior to acquiring the spectral data from the sample using reflection infrared spectroscopy.

28. The system of claim 21, wherein receiving spectral data from the sample comprises at least one of:
receiving spectral data from the sample in the absence of a drying treatment being applied to the sample; and
receiving spectral data from the sample in the absence of a reagent being applied to the sample for reducing water content of the sample.

29. (canceled)
